SEQUENCE LISTING (12 sequences)

SEQ ID NO: 1 - DNA, 25 bp, Bos taurus
ccacaacgccatcgagaaacgctac

SEQ ID NO: 2 - DNA, 25 bp, Bos taurus
ggccttccctgaccacccaacttag

SEQ ID NO: 3 - DNA, 84 bp, Bos taurus
cctggggccccagcctcctcgggcttggcagctctgttcagctgagcttcagggaagccccgggtggcacaggctcttccaggt

SEQ ID NO: 4 - DNA, 778 bp, Bos taurus
ccacaacgccatcgagaaacgctaccgctcttccatcaatgacaagatcgttgagctcaaggacctggtggtgggcaccgaggccaaggtgtgggctgaggccctaacagggtggctctgggcaagcaggcacctgggaggaggaggaggaagatggggctgggcagacagtcctggggccccagcctcctcgggcttggcagctctgttcagctgagcttcagggaagccccgggtggcacaggctcttccaggtgctggggatttagcaggggacagacagagatcctgctgttgagtagctgacgttctagcagggtgcgggggcgccagcagagctgggttggggtacgccaggtggcataagtgctgtgctggagagaagagggatgggggaagttgcaaacctaagttgggtggtcagggaaggcctcacaaaggtgatgcgatgtgtgaacagaggcctggaggaggtgagagggtgaggcataaagtggagtgatgttgacagcagccagccaggtgctgttgtctgtatcttacttacctgggcggcaggttctaggggaggaatgttcccagggagtaggaatagcagatgcaaatctcctgaggcgattgtgttgcaaagcagccctgccctctcgcttcagtgccaagctctctggcctgaccacttgtgccatctccctcccaccccacccctgcagttgaataaatctgccgtcttgcgcaaggccatcgactacatccgcttccttcagcacagcaaccagaa

SEQ ID NO: 5 - DNA, 3574 bp, Bos taurus
  CDS (22)..(3324)
ccgagaggaggtgactgcgccatggacgagccacccttcaacgaagcggccttggagctggcgctggccgagccgtgcgagctggacgcggcgctgctgaccgacatagcagacatgcttcagctcatcaacaaccaagacagcgaattcccgggcctattcgacccgccctacgctgggggcggagcagggaccacagaccctgccagtcctgacgccagctccccgggcagcctgtccccacctccttccacgatgagctcctcacttgaaggcttcctgggggtgaccaaggcgacacccccacccttgtcccctccccagcctgcgcccacccccctgaagatatacccatctgtgcctgccttctccccggtgcctgggatcaaggaggagccagcgcccctgactgtcctccagcccaccaccccccagcccctgcctggagctctcctgccgcagagtgttgcggccaccaccccaccgcagttcagctctgctcccattgtgggctaccccagccctccgggaggcttcacagcagggacccctccggggagcagctcgcagtcactggctggcctgccactggcttccctgccaggggtcccgcccgtctccttgcacagccaggttcagagcgcggccccccagcagctgttgacagccacagccacccccacggtggcccctggagcaaccgctgtgacctcccagatacagcaggtcccggtcctgctgcagccccacttcatcaaggcagactcgttgctcctgacgaccgtgaaaacagatgtgggagcccccttgaaagcggcaggcatccgctccctgggccctggcactgctgttcaggcagcgcccttgcagaccctggtgagtggcggggccatcctggccacggtgccactggtagtagacactgacaagctgcccatcaaccgacttgccagtggcaaggctccaggctcggcgcagagccgcggcgagaagcgtacagcccacaacgccatcgagaaacgctaccgctcttccatcaatgacaagatcgttgagctcaaggacctggtggtgggcaccgaggccaagttgaataaatctgccgtcttgcgcaaggccatcgactacatccgcttccttcagcacagcaaccagaagctcaagcaggagaacctgagtctgcgcactgctgtccacaaaagcaaatcgctgaaggacctggtgtcggcttgtggcagtggagggagcacagatgtgcccatggagggcatgaagccagaggtggtcgacaccctgagtccacccccctcagatgccggctcgccctcccagagcagccccttgtcccttggcagcaggggcagtagcagcggtggaagtggcagtgactcggagcccgacagcccggtctttgaggacggccaggtgaatccagagccgctgcctgctccccacagccagggcatgctggaccgctctcgcctggccctgtgcgcactcgtcttcctctgtctctcctgcaaccccttggcctccctgctgggtagccggggtcctgctggcccctccgacaccaccagcatcaaccacggtcccaggcgcagcatgctgggtgctgagggcagagatggtcctggctgggccccatggctgctgcccccactggtctggctgatgaatgggctgctggtgctcttctccttggcacttctctttgtctatggagaaccagtcactcggccccactcatgccccgccgtgcacttctggaggcatcgcaagcaggctgacctggacctggccaggggggactttgcccaggctgcccagcagctgtggctggccctgcgggccttgggccggcctctgcccacctcccacctggacctggcctgcagcctgctctggagcctcatccgccacctgctgcaacgtctctgggtgggccgctggctggccggctgggcagggggcctacggagggacagggccctacaggcagatgctcgcaccagtgcccgtgacgcagccctcgtctaccacaagctgcaccagctgcacaccatgggaaagtactcaggtgggcacctcgctgctgccaacctggcactgagtgccctgaacctggccgagtgcgctggagatgctgtgtccgtggccacgctggctgagatctacgtggccgccgcactcagggtcaaggccagtctgccccgggccttgcattttctgacacgcttcttcctgagcagtgctcgccaggcctgcctggcacagagcggctcagtgccccttgccatgcagtggctctgccaccctgtgggccaccgtttcttcgtggatggcaactgggccctgtgcagcgccccgagggacagcttgtacagcgtggctgggaacccagtggatcccctggcccaggtgactcaactgttccgcgaacatctcttggagcgagcactgaattgcgtggcccagcccagccccagtcctggatcagccgagggggacaaggagttctcagatgccctggggtacctgcagctgctgaacagctgttcggacgtggccggagctcttacctgcagcttctccatcagctccagcatggctgccacccccggcacagacccggtggccaagtggtgggcctctctgacagctgtggtgacccactggcttcggcgggatgaggaggcagccgagaggctgtacccattggtggagcacctgccccgtgccctgcaggagtccgagaaacccctacccagggcggctctgcactccttcaaggctgcccggaccattctaggccgcgggaaggctgagtctggcccagccagcctggcaatgtgtgagaaggccagtgggtacctgcaggacagcctggccaccacaccagctgacagctccattgacaaggccttgcagctgctcctgtgtgacctgctccttgtggcgcgcaccagcctctggcagcggcagaagctgccggcacccacccaggcctcgcagggccctggaggtggggcccaggcctctgccctcgagcttcgtggtttccagagggacctgagtggcctgaggcgtctggcacagaatgttcggcctgccatgcggagggtattcctacatgaagccactgcccgactgatggcaggggccagcccagcgcggacaaaacagcttctggatcgcagcctgaggaggagggtcggctcccgcactcggcgcgcggtggccaggatagtgctggcccctggtggccggctggggaattaccggggcctgccgccgccgccggatgtcaagtgctcccagtgacaacgctcctgggtgtcatgggccccagtccgcagctctccccctccctccagagagaagagaggggtgcatttcaccgaaccgagggaccctacccactgtgcccccctcccatctagggagacctgtgcatagtgtagatcggagtgcacagcctcctgggctcaagggtcaggcgttactttgcttttgcagactttatttttataggttgagaggtttgtaaaaaaaaaaaaaaaaaaaaaaaaaa

SEQ ID NO: 6 - protein, 1100 aa, Bos taurus
MDEPPFNEAALELALAEPCELDAALLTDIADMLQLINNQDSEFPGLFDPPYAGGGAGTTDPASPDASSPGSLSPPPSTMSSSLEGFLGVTKATPPPLSPPQPAPTPLKIYPSVPAFSPVPGIKEEPAPLTVLQPTTPQPLPGALLPQSVAATTPPQFSSAPIVGYPSPPGGFTAGTPPGSSSQSLAGLPLASLPGVPPVSLHSQVQSAAPQQLLTATATPTVAPGATAVTSQIQQVPVLLQPHFIKADSLLLTTVKTDVGAPLKAAGIRSLGPGTAVQAAPLQTLVSGGAILATVPLVVDTDKLPINRLASGKAPGSAQSRGEKRTAHNAIEKRYRSSINDKIVELKDLVVGTEAKLNKSAVLRKAIDYIRFLQHSNQKLKQENLSLRTAVHKSKSLKDLVSACGSGGSTDVPMEGMKPEVVDTLSPPPSDAGSPSQSSPLSLGSRGSSSGGSGSDSEPDSPVFEDGQVNPEPLPAPHSQGMLDRSRLALCALVFLCLSCNPLASLLGSRGPAGPSDTTSINHGPRRSMLGAEGRDGPGWAPWLLPPLVWLMNGLLVLFSLALLFVYGEPVTRPHSCPAVHFWRHRKQADLDLARGDFAQAAQQLWLALRALGRPLPTSHLDLACSLLWSLIRHLLQRLWVGRWLAGWAGGLRRDRALQADARTSARDAALVYHKLHQLHTMGKYSGGHLAAANLALSALNLAECAGDAVSVATLAEIYVAAALRVKASLPRALHFLTRFFLSSARQACLAQSGSVPLAMQWLCHPVGHRFFVDGNWALCSAPRDSLYSVAGNPVDPLAQVTQLFREHLLERALNCVAQPSPSPGSAEGDKEFSDALGYLQLLNSCSDVAGALTCSFSISSSMAATPGTDPVAKWWASLTAVVTHWLRRDEEAAERLYPLVEHLPRALQESEKPLPRAALHSFKAARTILGRGKAESGPASLAMCEKASGYLQDSLATTPADSSIDKALQLLLCDLLLVARTSLWQRQKLPAPTQASQGPGGGAQASALELRGFQRDLSGLRRLAQNVRPAMRRVFLHEATARLMAGASPARTKQLLDRSLRRRVGSRTRRAVARIVLAPGGRLGNYRGLPPPPDVKCSQ

SEQ ID NO: 7 - DNA, 26 bp, Bos taurus
gtgatgttccagaggaggtactacaa

SEQ ID NO: 8 - DNA, 25 bp, Bos taurus
aacgtttcatcccacaagcatagga

SEQ ID NO: 9 - DNA, 28 bp, Bos taurus
cctggtgtcctgttgttgtgcttcatcc

SEQ ID NO: 10 - DNA, 20 bp, Bos taurus
tcaactgttccgcgaacatc

SEQ ID NO: 11 - DNA, 23 bp, Bos taurus
catctgagaactccttgtccccc

SEQ ID NO: 12 - DNA, 25 bp, Bos taurus
tcttggagcgagcactgaattgcgt

(19) United States
(12) Patent Application Publication
(10) Pub. No.: US 2008/0010696 A1
(43) Pub. Date: Jan. 10, 2008

(54) Method of Determining Gene Relating to Favorable Beef Taste and Texture

(75) Inventors: Soichi Tsuji, Kobe-shi (JP); Hideyuki Mannen, Kobe-shi (JP)

Correspondence Address: KIRK HAHN, 14431 HOLT AVE, SANTA ANA, CA, 92705, US

(73) Assignee: THE NEW INDUSTRY RESEARCH ORGANIZATION, Kobe (JP)

(21) Appl. No.: 11/815,559

(22) PCT Filed: Feb. 3, 2006
(86) PCT No.: PCT/JP2006/301841

§ 371 Date: Aug. 4, 2007

(30) Foreign Application Priority Data
Feb. 4, 2005 (JP) 2005-028384

(51) Int. Cl. A01K 67/027; C07K 14/47; C12N 15/12; C12Q 1/68

(52) U.S. Cl. 800/15; 435/6; 530/350; 536/23.5

(57) ABSTRACT

By focusing attention on SREBP-1 gene, the relationship between this gene and the degree of unsaturation in beef body fat is examined. As a result, it is found out that this gene occurs as an “L-type” gene having a longer fifth intron and an “S-type” gene having a fifth intron shorter by 84 bases than the above one and these genotypes relate to the degree of unsaturation in beef body fat. A method of evaluating the content of unsaturated fatty acids in beef body fat based on these SREBP-1 genotypes is applicable to the evaluation of beef taste, texture and so on and, therefore, is industrially useful.

### TECHNICAL FIELD

The present invention relates to a method for evaluating quality of beef such as taste and texture through genotyping, and more specifically relates to a method for evaluating, on the basis of the genotype of sterol regulatory element binding protein (SREBP-1), whether or not it is a cattle, from which better quality of beef with better taste and texture can be produced. The present invention is useful for various fields such as livestock industry (feeding, breeding, reproduction, etc.), beef production, processing and distribution.

### BACKGROUND ART

It is known that the content of unsaturated fatty acid in beef fat is associated with quality of beef, such as the beef taste and texture. Generally, if the content of unsaturated fatty acid is high with low melting point, the beef is considered to have good quality, which gives good taste and texture.

However, in order to judge whether the beef has such quality, there was no other way but to depend on the subjective (sensuous) method, in which the beef was actually eaten and evaluated. In other words, there was no conventional method, which was more objective, simple and efficient, like the judgment approach of predicting quality of beef, simply based on the genotype of a specific bovine gene.

By the way, stearoyl-CoA desaturase (SCD) is known as an enzyme which desaturates beef fat. This enzyme desaturates stearoyl-CoA which plays an important role for in vivo biosynthesis of lipids and their degradation. Amino acid sequence and cDNA sequence of bovine stearoyl-CoA (derived from Bos taurus) are shown in DDBJ/EMBL/GenBank databases; Accession number “AB075020”.

The inventors disclose, in JP 2004-261014, a method for evaluating the amount of the unsaturated fatty acid content in beef fat, on the basis of a genotype of SCD, to evaluate whether or not it is a cattle, from which better quality of beef with better taste and texture can be produced.

As mentioned above, the content of unsaturated fatty acid in beef fat (in other words, the amount of the unsaturated fatty acid content) is associated with quality of beef, such as the beef taste and texture. If the amount of the unsaturated fatty acid content can be predicted on the basis of the genotype of a specific bovine gene, then it provides a new method which enables simple examination as to quality of beef, such as the beef taste and texture. Such method based on the genotype is useful not only for evaluation and selection of cattle (beef cattle) with good quality of beef, but also for breeding and reproduction of cattle.

Furthermore, the content of unsaturated fatty acid in beef fat is associated with the cholesterol accumulation caused by beef intake, and it is considered one of important features of the cattle, especially in the countries where beef is eaten very often, such as the Europe and the United States of America. Therefore, the above-mentioned method for evaluating the unsaturated fatty acid content based on the genotype, is also useful from the view point of health care.

The above-mentioned judgment method based on the SCD genotype, proposed by the inventors, is useful. However, the development of a new method for evaluating through a different genotype has been expected, because, in some cases, it was difficult to evaluate quality of beef only by the SCD genotype. Such new method can be expected to provide a more excellent evaluation method, by combining with the judgment method based on the SCD genotype.

### DISCLOSURE OF THE INVENTION

The object of the present invention is (1) to provide a new method for simply and efficiently evaluating the amount of the unsaturated fatty acid content in beef fat on the basis of the genotype of a bovine gene, and (2) to provide a new method for predicting, based on its evaluation, quality of beef, such as the beef taste and texture, more objectively.

The unsaturated fatty acid content is different among the cattle kinds. For example, the unsaturated fatty acid content of Japanese Black Cattle is higher than that of the Holstein-Friesian, and the melting point in beef fat of Japanese Black Cattle is lower. Therefore, Japanese Black Cattle is considered to have good quality, which gives good taste and texture. As a result of analysis, the inventors deduced that the SCD gene expression level may be associated with the difference of the unsaturated fatty acid content. Further analysis revealed that (1) there is a SRE (sterol regulatory element) sequence at the upstream regulatory region of SCD gene, to which SREBP-1 (sterol regulatory element binding protein) binds as a transcription factor, (2) the SREBP-1 gene expression level is associated with the SCD gene expression level, (3) there is a polymorphism in this SREBP-1 gene; i.e., one type (L type) having a longer fifth intron while another type (S type) having a fifth intron shorter by 84 bases than the above L type, and (4) some Japanese Black Cattle have the S type, and the unsaturated fatty acid content of cattle with this S type is higher and its melting point in beef fat is lower. We also clarified the gene sequence and the amino acid sequence of bovine SREBP-1, along with the above analysis. These findings led us to the present invention.

The present invention includes the following industrially useful inventions A) to L).

A) A method for evaluating the amount of the unsaturated fatty acid content in beef fat, on the basis of a genotype of sterol regulatory element binding protein (SREBP-1), to evaluate whether or not it is a cattle, from which better quality of beef with better taste and texture can be produced.

B) The method set forth in A), comprising a step (a) of determining the genotype of SREBP-1 by examination of a polymorphism in a fifth intron of this gene, and a step (b) of evaluating a cattle with a “S type” allele to produce better quality of beef (better taste and texture), compared with a cattle with a “L type” allele, said “S type” allele has a shorter fifth intron while said “L type” allele has a longer fifth intron.

C) The method set forth in B), comprising a step of amplifying a gene region including a deleted portion of 84 bases, related to the polymorphism in the fifth intron, by using a genomic DNA prepared from a test cattle as a template, and a step of determining the genotype of SREBP-1 by a length of an amplified fragment.

D) The method set forth in C), adopting a PCR (polymerase chain reaction) by use of a forward primer having a base sequence shown in SEQ NO. 1 and a reverse primer having a base sequence shown in SEQ NO.2 in the amplification step.

E) The method set forth in C), using a genomic DNA prepared from a hair of a test cattle as a template.

F) The method set forth in B), examining the polymorphism in the fifth intron by use of a gene polymorphism detector device such as a DNA chip.

G) A method for evaluating the amount of the unsaturated fatty acid content in beef fat, on the basis of a combination of a genotype of sterol regulatory element binding protein (SREBP-1) and a genotype of stearoyl-CoA desaturase (SCD), to evaluate whether or not it is a cattle, from which better quality of beef with better taste and texture can be produced

H) The method set forth in G), comprising a step of evaluating a cattle to produce better quality of beef (better taste and texture), if the cattle has three or more alleles, as a total number of a number of “S type” alleles among the SREBP-1 genotype and a number of “A-type” alleles among the SCD genotype.

I) A bovine-derived SREBP-1 protein indicated by (a) or (b) below:
(a) a protein having an amino acid sequence of SEQ NO.6;
(b) a protein having an amino acid sequence obtained by deletion, replacement, and/or addition of one or several amino acids in the amino acid sequence of SEQ NO.6 and having a transcriptional regulatory activity by binding a SRE sequence.

J) A bovine-derived SREBP-1 gene, encoding any one protein set forth in I).

K) The gene set forth in J), said gene being a DNA indicated by (a) or (b) below:

(a) a DNA having a base sequence ranging from the 22nd base to the 3324th base, among a base sequence of SEQ NO.5;

(b) a DNA that has a base sequence hybridized under stringent conditions to a DNA having a base sequence that is complementary to at least 100 base sequence of SEQ NO.5, and that encodes a protein having a transcriptional regulatory activity by binding a SRE sequence.

L) A transgenic cattle, produced by introducing the gene set forth in K).

### EFFECT OF THE INVENTION

According to the present invention, it is possible to evaluate not only the unsaturated fatty acid content, but also quality of beef such as taste and texture, by examining whether the SREBP-1 genotype is L type or S type. One example of easy examination is a PCR method using a sample prepared from a cattle hair, in order to determine the cattle has L type or S type. More excellent judgment is available by combining the judgment method of the present invention with the judgment method based on the SCD genotype.

The unsaturated fatty acid content in beef fat is associated with not only quality of beef, but also the cholesterol accumulation caused by beef intake, and it is considered one of important features of the cattle, especially in the countries where beef is eaten very often, such as the Europe and the United States of America. The present invention is useful for various fields such as identification of cattle, selection of stud bull, livestock industry (feeding, breeding, reproduction, etc.), beef production, processing and distribution.

### BRIEF DESCRIPTION OF THE DRAWINGS

FIG. 1 is a graph showing the relation between the SCD gene expression level and the content of mono-unsaturated fatty acid (MUFA) in the intermuscular adipose tissue.

FIG. 2 is a graph showing the result of examining a time change in the SCD gene expression level of each cattle kind.

FIG. 3 is a graph showing the result of examining the relation between the MUFA content and the SCD gene expression level, about plural samples of each cattle kind, which were collected at different time.

FIG. 4 is a figure showing a transcriptional regulatory sequence in the upstream regulatory region of SCD gene.

FIG. 5 is a graph showing the relation between the SCD gene expression level and the SREBP-1 gene expression level.

FIG. 6 is a figure explaining a genomic structure of SREBP-1 gene and a deleted portion of the fifth intron.

FIG. 7 is a figure showing the result of judging the SREBP-1 genotype, about each cattle kind of Japanese Black Cattle and the Holstein-Friesian.

FIG. 8 is a figure showing the result of judging the SREBP-1 genotype, and measuring the unsaturated fatty acid content and the melting point of each genotype.

### SEQUENCE LISTING FREE TEXT

SEQ NO.1: Forward primer sequence for typing bovine SREBP-1 gene.

SEQ NO.2: Reverse primer sequence for typing bovine SREBP-1 gene.

SEQ NO.3: Sequence of a deleted portion in the fifth intron of bovine SREBP-1 gene.

SEQ NO.4: Sequence ranging over the fifth exon, the fifth intron and the sixth exon of bovine SREBP-1 gene.

SEQ NO.5: cDNA sequence and amino acid sequence of bovine SREBP-1.

SEQ NO.6: Amino acid sequence of bovine SREBP-1.

SEQ NO.7: Forward primer sequence for real-time PCR of bovine SCD mRNA.

SEQ NO.8: Reverse primer sequence for real-time PCR of bovine SCD mRNA.

SEQ NO.9: TaqMan™ probe sequence for real-time PCR of bovine SCD mRNA.

SEQ NO.10: Forward primer sequence for real-time PCR of bovine SREBP-1 mRNA.

SEQ NO.11: Reverse primer sequence for real-time PCR of bovine SREBP-1 mRNA.

SEQ NO.12: TaqMan™ probe sequence for real-time PCR of bovine SREBP-1 mRNA.

### BEST MODE FOR CARRYING OUT THE INVENTION

Specific embodiments of the present invention are described in detail below with reference to the drawings.

### (1) The Judgment Method of the Present Invention

The present invention is a method for evaluating the amount of the unsaturated fatty acid content in beef fat, based on the genotype of SREBP-1, to evaluate whether or not it is a cattle, from which better quality of beef with better taste and texture can be produced.

Here, “cattle” means the animal belonging to Genus Bos, especially livestock cattle (Bos primigenius). The Europeane cattle (Bos taurus) and the Indian cattle (Bos indicus) are included in this “livestock cattle”, and the following breeds of cattle are included in this “Europeane cattle (Bos taurus)”; i.e., Japanese cattle (such as Japanese Black, Japanese Brown, Japanese Polled and Japanese Shorthorn) and Europeane cattle (such as the Holstein-Friesian, the Jersey, the Shorthorn for meat, the Hereford and the Aberdeen Angus). The “cattle” used as a test animal (source of specimen) may be any one of the above beef cattle.

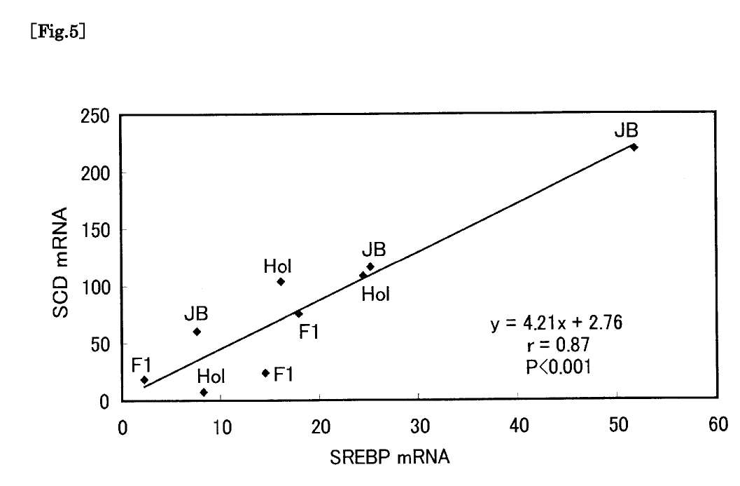

“SREBP-1 (sterol regulatory element binding protein)” is a transcription factor derived from cattle, which binds a SRE sequence (see FIG. 4) at the upstream of SCD gene and has a transcriptional regulatory activity. The cDNA sequence of SREBP-1 gene is shown in SEQ NO.5, which was isolated from Japanese Black and decided by the inventors, together with the amino acid sequence encoded by the open reading frame (ORF) of this gene. The amino acid sequence of SREBP-1 protein is also shown in SEQ NO.6.

FIG. 6 schematically shows a genomic structure of the above SREBP-1 gene. As shown, the SREBP-1 gene comprises 19 exons and 18 introns. In a base sequence of the fifth intron (intron 5), we have found a polymorphism; i.e., one genotype having the underlined 84 bases of FIG. 6 while another genotype losing these 84 bases. Hereafter, the underlined 84 bases are conveniently called a “deleted portion”. Also, one genotype having a longer fifth intron because of having this deleted portion, is called a “L type” while another genotype having a shorter fifth intron because of losing this deleted portion, is called a “S type”. The deleted portion is ranging from the 85th base to the 168th base, in the base sequence of the fifth intron of the “L type”.

Thus, it was found that the genotype of bovine SREBP-1 gene can be roughly classified into two types of “L type” and “S type”. Further investigation revealed that the genotype of bovine SREBP-1 gene is associated with fatty acid composition (in other words, “the content of unsaturated fatty acid in beef fat” or “the amount of the unsaturated fatty acid content”). That is, it was found that there is a significant difference between the above “L type” and “S type” in the content of unsaturated fatty acid in beef fat. Although details of the investigation will be explained in the Example as described later, the percentage of mono-unsaturated fatty acid (MUFA) content in beef fat was the highest when the genotype of bovine SREBP-1 gene was (S/S) having homozygous S type. When the genotype of bovine SREBP-1 gene was (L/S) having heterozygous L and S type, the percentage was higher than that in the case of the genotype (L/L) having homozygous L type (see FIG. 8).

As mentioned above, the content of unsaturated fatty acid in beef fat is associated with quality of beef such as taste (flavor) and texture. In general, if the content of unsaturated fatty acid is high, then the melting point of beef fat will be low (FIG. 8), and the beef is considered to have good quality, which gives good taste and texture. Therefore, if the genotype of bovine SREBP-1 gene is (S/S), such cattle with (S/S) can be evaluated (estimated) to produce better quality of beef (better taste and texture), compared with the cattle with (L/L). Thus, by examining the genotype of bovine SREBP-1 gene, it becomes possible not only to judge the content of unsaturated fatty acid in beef fat, but also to judge whether it is the cattle, from which high quality of beef (with better taste and texture) can be produced.

In the method of the present invention, the method for examining the genotype of bovine SREBP-1 gene should be not limited, and various known methods can be applied to the method for directly, or indirectly, examining the presence or absence of the above-mentioned “deleted portion”. The PCR (Polymerase Chain Reaction) method is an easy and simple method with good accuracy, to examine the presence or absence of the “deleted portion”, and to decide the genotype of bovine SREBP-1 gene. The following is a brief explanation of the judgment using this PCR method.

### (2) Method for Judging the SREBP-1 Genotype by the PCR Method

Genetic sample (specimen) for the judgment may be a genomic DNA. The genomic DNA can be extracted and purified by a known method from any organ, tissue or cell of the test cattle (origin of specimen). Extraction of the organ, tissue or cell from the test cattle may be carried out before slaughter, or after slaughter. The cell from the test cattle may be blood (the cell in blood), the cell in the amniotic fluid, or the cultured cell (obtained from extracted tissue etc.). The genomic DNA was prepared from the blood in the Example as described later. The extraction and purification of the genomic DNA may be simplified or omitted, if a target gene region can be amplified by the PCR method without such step.

The genomic DNA prepared by the above method may be used as a template in the PCR method, for the purpose of determination of the genotype (i.e., genotyping) by examining the presence or absence of the above deleted portion. After amplifying a target region including the deleted portion by such PCR method, the genotype of bovine SREBP-1 gene can be judged (determined) according to a length of the amplified fragment.

The above PCR method should be not limited about each condition, used reagents and primers, etc. The following is an explanation about one example of the PCR method, which was adopted in the Example as described later.

The composition of the PCR solution was as follows; genomic DNA 20 ng, TaKaRa Ex Taq™ polymerase HS 0.5 Unit, 10×Taq polymerase buffer 2.0 μl, 25 mM dNTP mix 1.6 μl, each 1.0 μl of forward primer (10 pmol) and reverse primer (10 pmol), and ultrapure water added to be total 20 μl of solution.

Designed sequences of forward primer and reverse primer are shown in the following (a) and (b) respectively.

| | | |
|---|---|---|
| (a) | 5′- CCACAACGCCATCGAGAAACGCTAC -3′ | (SEQ No. 1) |
| (b) | 5′- GGCCTTCCCTGACCACCCAACTTAG -3′ | (SEQ No. 2) |

The forward primer sequence was designed based on a specific base sequence in the fifth exon (corresponding to 1 to 25 of SEQ No.4), which is at the upstream of the deleted portion, while the reverse primer sequence was designed based on a specific base sequence in the fifth intron (corresponding to 408 to 432 of SEQ No.4), which is at the downstream of the deleted portion. The deleted portion is ranging from 173 to 256 of SEQ No.4.

The PCR was carried out by the following condition; firstly (1) 94° C. for 2 minutes, next (2) 30 cycles of 94° C. for 30 seconds/65° C. for 30 seconds/72° C. for 1 minute, followed by (3) 72° C. for 7 minutes.

Different amplified fragments (PCR products) with different length are obtained by the above PCR method, according to the presence or absence of the deleted portion. The genotype of a test cattle (specimen) can be easily judged whether it is a L type or a S type, for example by electrophoresis of its PCR product with a marker.

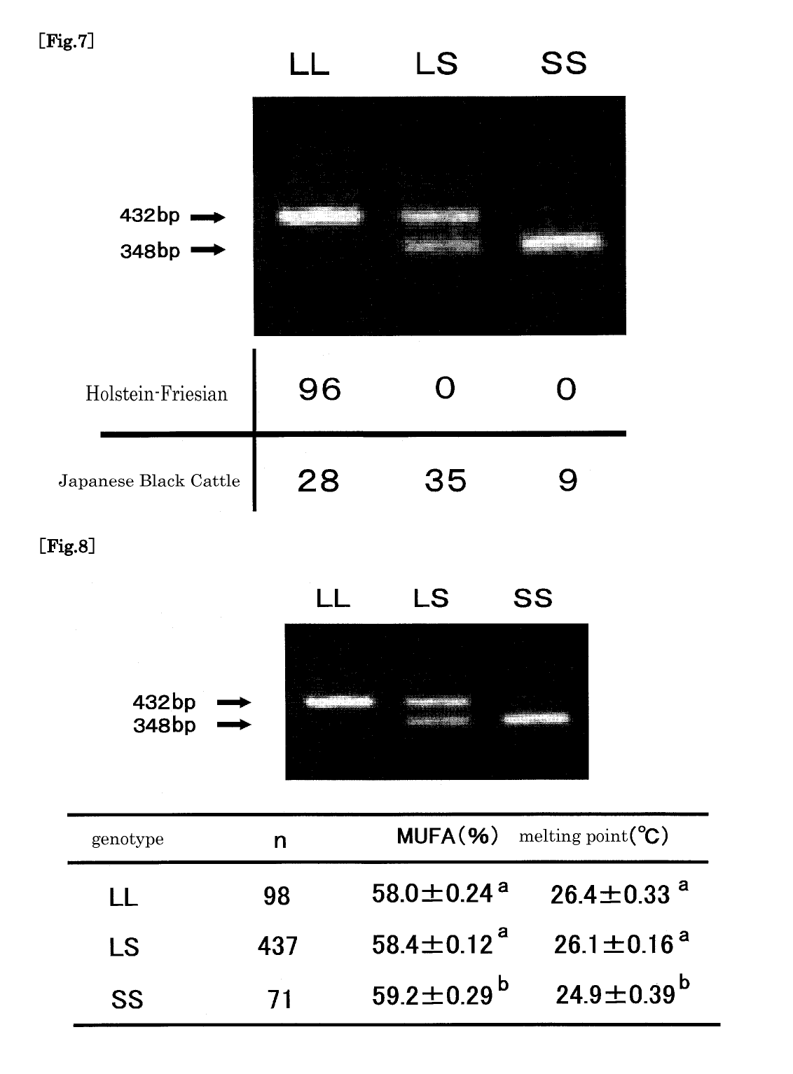

FIGS. 7 and 8 show a result of the electrophoresis. As shown in these Figures, when only longer DNA band of 432 bp appeared, then the genotype of a test cattle is judged the type (LL) having homozygous L type. When only shorter DNA band of 348 bp appeared, then the genotype is judged the type (SS) having homozygous S type. When both of shorter and longer DNA bands appeared, then the genotype is judged the type (LS) having heterozygous L and S type. Thus, this PCR method enables simple and precise judgment of the genotype of bovine SREBP-1 gene.

### (3) Other Embodiments of the Present Invention

The method of the present invention is not limited to the PCR method described in the above (2). For example, each condition for reaction, used reagents and primers, etc. can be variously changed, even when using the PCR method.

Of course, in addition to the PCR method, other methods may be used for the judgment (method) of the present invention. One example is a method by use of gene polymorphism detector tool (device) such as a DNA chip. This method is characterized by detection of hybridization signal between a probe and a genetic sample prepared from a test animal, using the DNA chip (or, similar device) that comprises, on a substrate, the probe to detect the presence or absence of the deleted portion. The probe can be designed based on a base sequence in the deleted portion, or its complementary sequence. Here, the word “DNA chip” principally means a synthesized-type DNA chip, comprising a synthesized oligonucleotide as the probe, but also includes an attached-type DNA microarray, comprising attached cDNA such as PCR product as the probe. The gene polymorphism detector tool, such as the DNA chip, is available to the method of the present invention, and also included in the present invention.

Other methods of the present invention include a method for detecting the presence or absence of the deleted portion, for example by PCR-RFLP (Restriction Fragment Length Polymorphism) method, and an amplification method other than the PCR, such as RCA method. In the PCR-RFLP method, a restriction enzyme may be used to cut a base sequence within the deleted portion. Genotyping is also possible by direct sequencing of amplified fragment using a sequencer, after DNA amplification.

The method of preparing a genetic sample from a test cattle is not limited to a specific method. It is a preferred approach to prepare a genetic sample from a hair of a test cattle because the cattle has no damages.

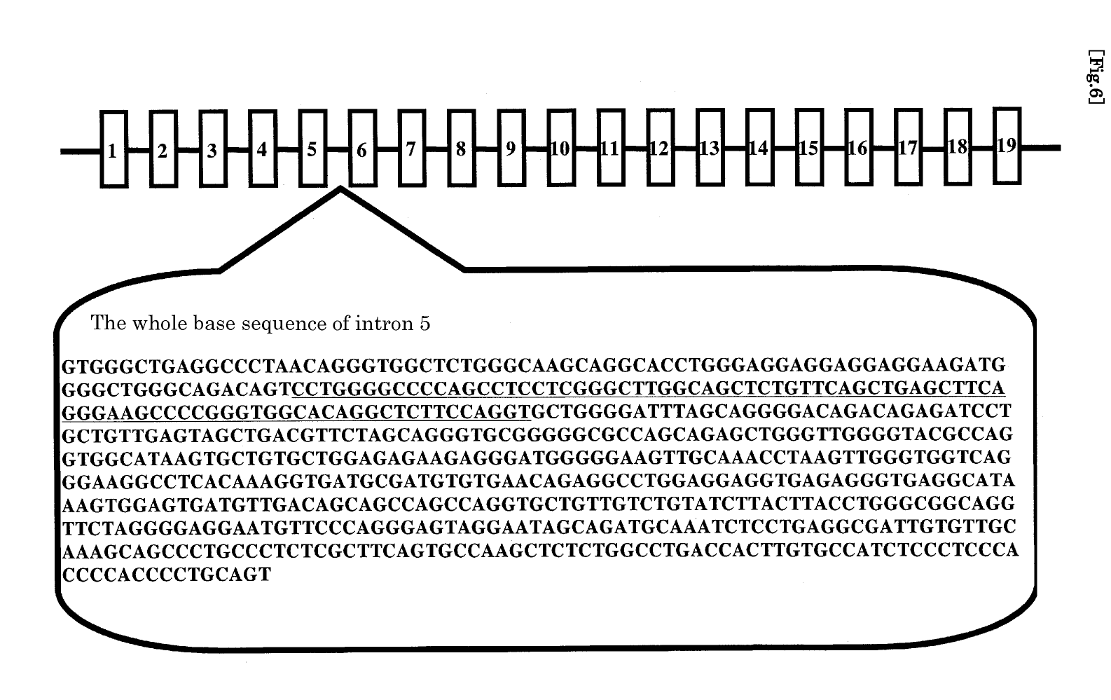

In addition to the above polymorphism in the fifth intron, other polymorphism (mutation) may have happened on the base sequence of bovine SREBP-1 gene, among animals belonging to Genus Bos (even among animals belonging to the Europeane cattle (Bos taurus)). Such polymorphism (mutation) on SREBP-1 gene may be associated with the unsaturated fatty acid percentage in beef fat, because the SREBP-1 gene expression level was correlated with the SCD gene expression level (FIG. 5). The prediction of quality of beef, as well as the prediction of the unsaturated fatty acid content, may be available, by investigating such polymorphism (mutation). For example, when such polymorphism (mutation) is on the ORF (open reading frame) of SREBP-1 gene, then cDNA may be prepared from mRNA, in order to detect its polymorphism (mutation). When such polymorphism (mutation) is on the regulatory region at the upstream of the gene, then it is possible to know an effective genotype by comparing genomic sequences of that region.

### (4) Industrial Applicability (Utility) of the Present Invention

As mentioned above, the present invention is useful for various fields such as identification of cattle, selection of stud bull, livestock industry (feeding, breeding, reproduction, etc.), beef production, processing and distribution. For example, the present invention can be used for evaluation of beef quality from the beef cattle such as Japanese Black cattle. Moreover, the present invention can be used, through the above evaluation, for breeding and reproduction. That is, the present invention may be applied to the breeding strategy to produce a superiol herd which has the characteristics of high quality beef producer.

The unsaturated fatty acid content in beef fat is also associated with the cholesterol accumulation caused by beef intake. The present invention enables prediction of the unsaturated fatty acid content and therefore, it is also useful from the view point of human health. For example, development of the beef with lower melting point is important for production and development of a healthy beef (good beef for health), especially in the countries where beef is eaten very often, such as the Europe and the United States of America.

The present invention is available to various fields such as animal science and livestock industry, through selection and reproduction of a target cattle or reproduction by using sperm or fertilized egg, etc. For example, the present invention is available to reproduction using recombinant DNA technology etc., as well as various gene experiments. Also, the present invention is applicable to prenatal diagnosis (judgment) for reproduction. Such prenatal diagnosis includes the method to pick up embryo cell from the amniotic fluid of the uterus, to prepare genetic samples and to prepare selected eggs which will produce high quality beef.

More excellent judgment is available by combining the judgment method of the present invention with the judgment method based on the SCD genotype (for example, see Table 3 described later). In this method, if a cattle has both “S type” of the SREBP-1 genotype and “A type” of the SCD genotype (see JP 2004-261014), then its cattle can be evaluated to have good quality of beef, since the unsaturated fatty acid percentage in beef fat is higher and the melting point is lower. Especially, if a cattle has three or more alleles, as a total number of a number of S types and a number of A types, then its melting point in beef fat tends to be remarkably lower, and its unsaturated fatty acid percentage tends to be higher.

### (5) Bovine SREBP-1 Gene and Protein

Bovine SREBP-1 gene and protein are a new gene and protein, which were cloned and identified for the first time by the inventors and therefore, these also fall within the scope of the present invention.

The SREBP-1 protein of the present invention includes (a) a protein having an amino acid sequence of SEQ NO.6, and (b) a protein having an amino acid sequence obtained by deletion, replacement, and/or addition of one or several amino acids in the amino acid sequence of SEQ NO.6 and having a transcriptional regulatory activity by binding a SRE sequence.

“Bovine SREBP-1 protein” of the present invention may have additive polypeptides. An example of such additive polypeptides includes a case of having an epitope such as His, Myc, or flag.

The “deletion, replacement, and/or addition of one or several amino acids” means the deletion, replacement, and/or addition of a number of amino acids that can be deleted, replaced, and/or added by a conventional method such as site-specific mutagenesis. That is, the protein of (b) is a mutant protein of the protein of (a), and here “mutant” is used primarily to mean mutation artificially introduced by a conventional method, but it also includes similar naturally-occurring mutant proteins isolated and purified.

The SREBP-1 gene of the present invention, encoding the SREBP-1 protein of the present invention, includes (a) a DNA having a base sequence ranging from the 22nd base to the 3324th base, among a base sequence of SEQ NO.5, and (b) a DNA that has a base sequence hybridized under stringent conditions to a DNA having a base sequence that is complementary to at least 100 base sequence (preferably over 200 base sequence, more preferably over 500 base sequence) of SEQ NO.5, and that encodes a protein having a transcriptional regulatory activity by binding a SRE sequence.

“Gene” of the present invention is preferably cDNA, but it can also be RNA or genomic DNA. Furthermore, “gene” of the present invention may include a sequence other than coding region of bovine SREBP-1 protein, such as a sequence for untranslated region (UTR) or vector sequence (including expression vector sequence).

Hybridization under stringent conditions can be carried out using for example the method set forth in “Molecular Cloning: Cold Spring Harbor Laboratory Press, Current Protocols in Molecular Biology”; Wiley Interscience, and more specifically the following method is exemplified.

DNA molecules from a cDNA library or the like are transferred to a membrane and hybridized with labeled probes within a hybridization buffer. The composition of the hybridization buffer is for example 0.1 wt % SDS, 5 wt % dextran sulfate, 1/20 content blocking reagent, and 2 to 7×SSC. One example for the blocking reagent is to use a solution of 100× Denhardt's solution, 2% (weight/volume) Bovine serum albumin, 2% (weight/volume) polyvinyl pyrrolidone prepared at five times concentration and then diluted to 1/20 concentration.

The hybridization temperature is in the range of 40 to 80° C., more preferably 50 to 70° C., and most preferably 55 to 65° C., and after incubation of between several hours and overnight, the membrane is washed with a washing buffer. The washing temperature is preferably room temperature or the temperature during hybridization. The washing buffer composition is preferably a 0.1 to 6×SSC+0.1 wt % SDS solution, and the membrane is washed several times with the washing buffer while altering the SSC concentration. The DNA hybridized with the probe is then detected using the label attached to the probe.

The gene and protein of the present invention can be used widely for the research of the cattle, and can be also used for the production of a transgenic cattle. Such transgenic cattle can be produced with a known transgenic technology (for example, Science 1998 Oct. 23; 282(5389): 751-4), which comprises a step of introducing bovine SREBP-1 of the present invention into a cellular nucleus, a step of making this nucleus fuse together with a bovine egg, and a step of transplanting thus obtained embryo into a surrogate mother.

### EXAMPLE

The present invention is described in detail below through Example thereof, but in no way is the present invention limited to this Example.

### Example 1

Relation Between the Content of Unsaturated Fatty Acid and the SCD Gene Expression Level

The content of unsaturated fatty acid is different among the cattle kinds, and as for Japanese Black Cattle, the content of unsaturated fatty acid is higher than that of the Holstein-Friesian, and the melting point in beef fat of Japanese Black Cattle is lower. We investigated a relation between the content of unsaturated fatty acid and the expression level of bovine SCD gene, in order to examine a possibility that the SCD gene expression level may be associated with the difference of the unsaturated fatty acid content among the cattle kinds.

After bovine mRNA was extracted and purified from cattle's adipose tissue or muscular tissue according to a conventional method, cDNA was synthesized with a reverse transcriptase. Using this cDNA as a template, real-time PCR was carried out with TaqMan™ probe, in order to measure the SCD gene expression level (the amount of mRNA). The used sequences of a forward primer, a reverse primer and a TaqMan™ probe are respectively shown in the following (1), (2) and (3).

| | | |
|---|---|---|
| (1) | 5′- GTGATGTTCCAGAGGAGGTACTACAA -3′ | (SEQ No. 7) |
| (2) | 5′- AACGTTTCATCCCACAAGCATAGGA -3′ | (SEQ No. 8) |
| (3) | 5′- CCTGGTGTCCTGTTGTTGTGCTTCATCC -3′ | (SEQ No. 9) |

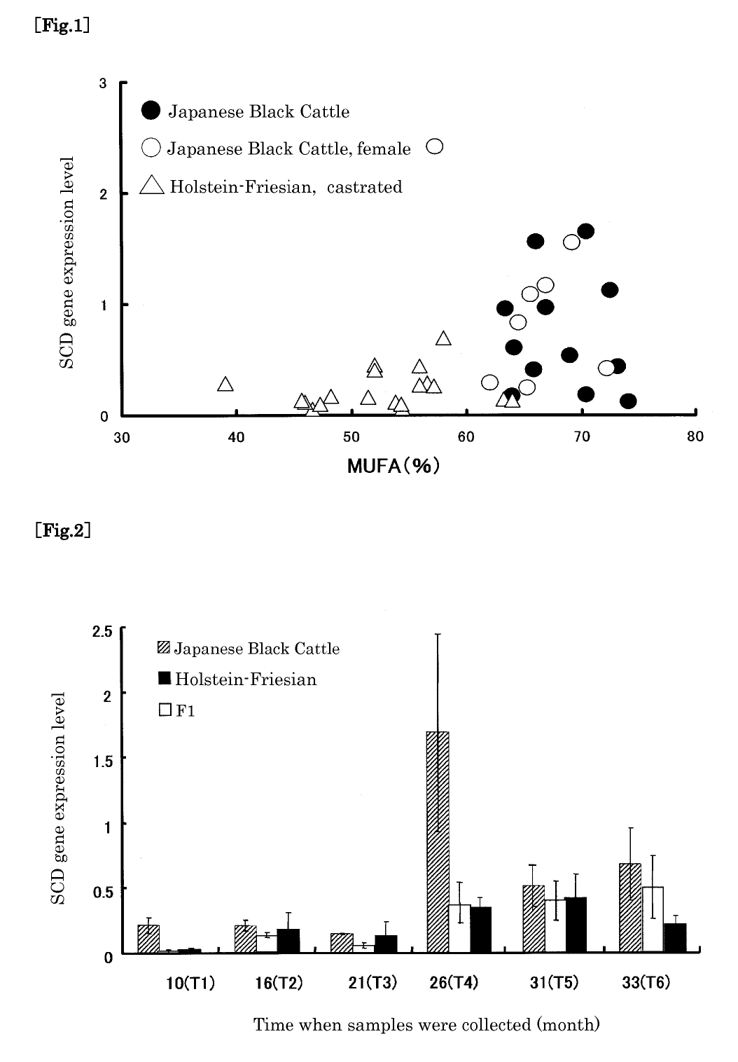

The result of the above experiment is shown in the graph of FIG. 1, showing the relation between the SCD gene expression level (relative value) and the content (%) of mono-unsaturated fatty acid (MUFA) in the intermuscular adipose tissue. Also, Table 1, listed below, shows the investigation result of the amount of SCD mRNA and the MUFA content, in the muscle collected from the neck at the time of slaughter. The amount of SCD mRNA and the MUFA content were different between the cattle kinds. Little letters “a” and “b” in Table 1 (and Table 2 described later) show the significant difference by the risk 5% or less.

TABLE 1

| Kind | n | SCD mRNA | MUFA (%) |
|---|---|---|---|
| Japanese Black Cattle, castrated | 12 | 0.72 ± 0.15 ^{a} | 64.5 ± 1.0 ^{a} |
| Japanese Black Cattle, female | 8 | 1.00 ± 0.26 ^{a} | 61.4 ± 1.4 ^{a} |
| Holstein-Friesian, castrated | 19 | 0.23 ± 0.04 ^{b} | 50.0 ± 1.3 ^{b} |

^{a b} <0.05

The result of the above experiment suggests that the SCD gene expression level is associated with the difference of the unsaturated fatty acid content among the cattle kinds. Furthermore, we carried out the following experiment in which different cattle kinds of the same month age were compared.

We investigated the MUFA content and the SCD gene expression level (the amount of mRNA) in both the muscle and the subcutaneous adipose tissue by using the samples, which were collected from the fattening cattle of 26 months of each kind of “Japanese Black Cattle”, “Holstein-Friesian” and “F1 (hybrid)” by the biopsy method. Table 2, listed below, shows the result.

TABLE 2

| | | SCD mRNA | | |
|---|---|---|---|---|
| Kind | n | muscle | adipose tissue | MUFA (%) |
| Japanese | 3 | 3.3 ± 1.0 ^{a} | 132.1 ± 34.1 ^{a} | 57.3 ± 0.6 ^{a} |
| Black Cattle | | | | |
| F1 | 3 | 1.1 ± 0.3 ^{b} | 73.5 ± 22.7 ^{a b} | 54.0 ± 0.4 ^{a b} |
| Holstein-Friesian | 3 | 0.8 ± 0.2 ^{b} | 39.5 ± 12.9 ^{b} | 53.4 ± 1.6 ^{b} |

^{a b} P < 0.05

In each cattle kind, the SCD gene expression level was higher in the adipose tissue than in the muscle, and it was associated with the MUFA content.

FIG. 2 shows the result of examining a time change of the SCD gene expression level of each cattle kind. As shown in FIG. 2, the SCD gene expression level of Japanese Black Cattle was especially high, and significantly different, at the month ages of 10 months and 26 months when the samples were collected.

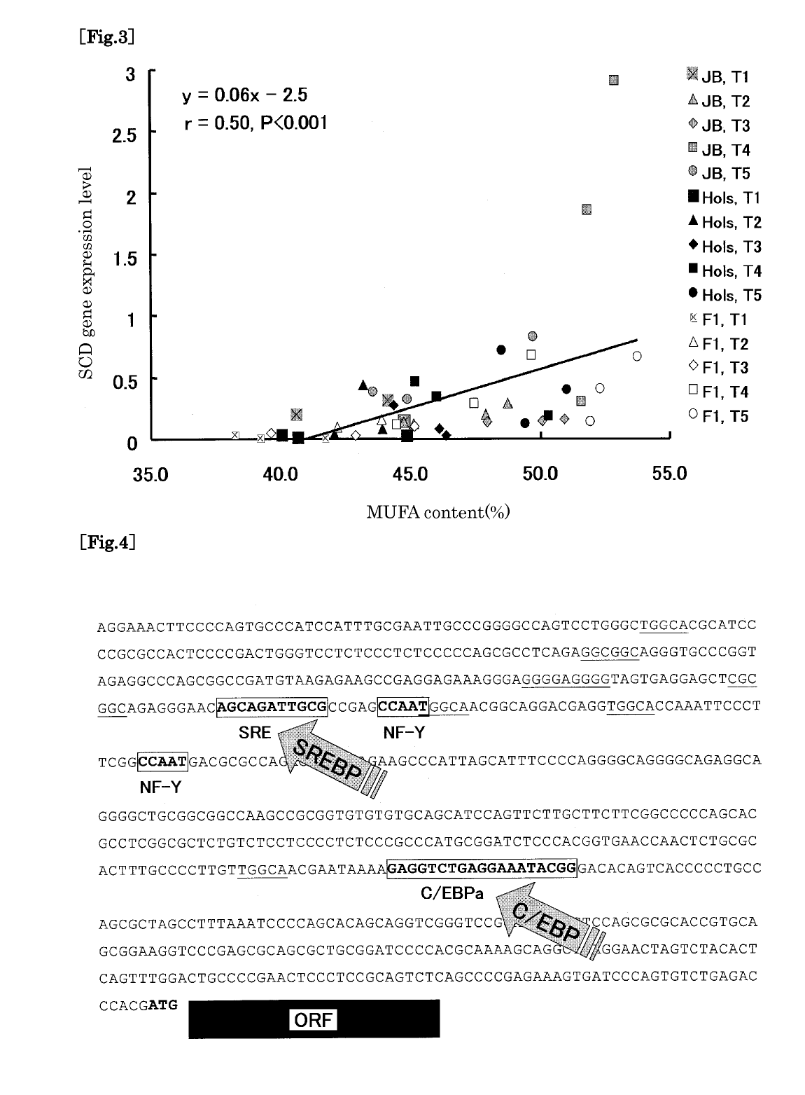

FIG. 3 shows the result of examining the relation between the MUFA content and the SCD gene expression level, about each kind of Japanese Black Cattle (JB), the Holstein-Friesian (Hols) and the F1 hybrid (F1). The samples of each kind were collected at the month ages of 10, 16, 21, 26 and 31 months, which are respectively shown as T1-T5 in FIG. 3.

The results of the above-mentioned experiments suggest that the SCD gene expression level is associated with the difference of the unsaturated fatty acid content.

### Example 2

Relation Between the SCD Gene Expression Level and the SREBP-1 Gene Expression Level

We paid attention to the SREBP-1 gene, as a factor affecting the SCD gene expression level, because of the following reasons; (1) as shown in FIG. 4, there is a SRE sequence at the upstream of SCD gene, and the SREBP-1 is considered to bind to this SRE sequence and have an activity of regulating transcription of SCD gene; (2) there is a QTL (Quantitative Trait Loci), that is related to body fat, in the chromosomes 19 and 26, and SCD gene is located in the chromosome 26 while SREBP-1 gene is located in the chromosome 19. This suggests that SREBP-1 may be mainly involved in the control of the SCD gene expression.

In order to examine the relation between the SCD gene expression level and the SREBP-1 gene expression level (the amount of mRNA), real-time PCR was carried out similarly to the above-mentioned real-time PCR. The sequences of a forward primer, a reverse primer and a TaqMan™ probe, used to measure SREBP-1 mRNA, are respectively shown in the following (4), (5) and (6).

| | | |
|---|---|---|
| (4) | 5′- TCAACTGTTCCGCGAACATC -3′ | (SEQ No. 10) |
| (5) | 5′- CATCTGAGAACTCCTTGTCCCCC -3′ | (SEQ No. 11) |
| (6) | 5′- TCTTGGAGCGAGCACTGAATTGCGT -3′ | (SEQ No. 12) |

As a result of the above experiment, the amount of SCD mRNA was associated with the amount of SREBP-1 mRNA, as shown in FIG. 5, and this result suggests that the SREBP-1 gene expression level may affect the SCD gene expression level.

### Example 3

Relation Between the MUFA Content and the Polymorphism Found on SREBP-1 Gene

We investigated the presence of any mutation on SREBP-1 gene. As a result, we found out a polymorphism in the fifth intron (intron 5) between one genotype (L type) having the underlined 84 bases of FIG. 6 while another genotype (S type) losing these 84 bases.

FIG. 7 shows the result of judging the above SREBP-1 genotype, about each cattle kind of Japanese Black Cattle and the Holstein-Friesian. The genotype was judged by the above-mentioned PCR. Used reagents and condition for reaction, etc. are described above, and here omitted explanation thereof. As shown in FIG. 7, all the Holstein-Friesians had only the L type in this investigation.

FIG. 8 shows not only the result of determining the genotype of each test cattle by the above judgment method, but also the table summarizing the result of measuring the unsaturated fatty acid content and the melting point of each genotype. As shown in this table, the unsaturated fatty acid percentage in beef fat was the highest in the genotype (SS) and it was higher in the genotype (LS) than the genotype (LL), which is a result of analysis of variance in one factor of the SREBP-1 genotype. This table also shows the comparison of melting point. This result supports that, when the unsaturated fatty acid percentage is higher, the beef melting point is lower and the beef may have better quality, which gives better taste and texture.

### Example 4

Judgment by the Combination of SREBP-1 Genotype and SCD Genotype

About each test cattle, SREBP-1 genotype was decided according to the above judgment method, while SCD genotype was decided according to the method described in JP2004-261014. In addition, we investigated the relation among the combination of these both genotypes, the unsaturated fatty acid content, and the melting point. The result is shown in Table 3.

TABLE 3

| genotype | n | MUFA(%) | melting point(° C.) |
|---|---|---|---|
| LL/AA | 24 | 58.5 ± 0.48 | 26.0 ± 0.66 |
| LL/AV | 67 | 58.0 ± 0.29 | 26.4 ± 0.39 |
| LL/VV | 7 | 56.3 ± 0.89 | 28.4 ± 1.22 |
| LS/AA | 99 | 59.3 ± 0.89 | 24.9 ± 0.32 |
| LS/AV | 309 | 58.2 ± 0.13 | 26.3 ± 0.18 |
| LS/VV | 29 | 57.3 ± 0.44 | 27.3 ± 0.60 |
| SS/AA | 20 | 59.2 ± 0.53 | 24.3 ± 0.72 |
| SS/AV | 42 | 59.6 ± 0.36 | 24.7 ± 0.50 |
| SS/VV | 9 | 57.2 ± 0.78 | 27.3 ± 1.10 |

Based on this result, it can be evaluated that, if a cattle has a combination of “S type” as SREBP-1 genotype and “A type” as SCD genotype, then its cattle will produce better quality of beef. Especially, if a cattle has three or more alleles, as a total number of a number of “S type” alleles of SREBP-1 genotype and a number of “A-type” alleles of SCD genotype, then its cattle can be evaluated to produce better quality of beef, with higher unsaturated fatty acid content and lower melting point.

### INDUSTRIAL APPLICABILITY

As mentioned above, the present invention is a method for evaluating quality of beef such as taste and texture, on the basis of the genotype of sterol regulatory element binding protein (SREBP-1), and useful for various fields such as identification of cattle, selection of stud bull, livestock industry (feeding, breeding, reproduction, etc.), beef production, processing and distribution.

1. A method for evaluating the amount of the unsaturated fatty acid content in beef fat comprising;
determining a genotype of sterol regulatory element binding protein (SREBP-1) to evaluate an animal of Genus Bos for better quality of beef with better taste and texture.

2. The method of claim 1, where the determining the genotype of SREBP-1 is by examination of a polymorphism in fifth intron of SREBP-1, the polymorphism being a “S type” allele, which has a shorter fifth intron, and a “L type” allele, which has a longer fifth intron, and selecting the animal of Genus Bos with a “S type” allele.

3. The method of claim 2, comprising a step of amplifying a gene region including a deleted portion of 84 bases, related to the polymorphism in the fifth intron, by using a genomic DNA prepared from a test animal of Genus Bos as a template, and a step of determining the genotype of SREBP-1 by a length of an amplified fragment.

4. The method of claim 3, adopting a PCR (polymerase chain reaction) by use of a forward primer having a base sequence shown in SEQ NO. 1 and a reverse primer having a base sequence shown in SEQ NO. 2 in the amplification step.

5. The method of claim 3, using a genomic DNA prepared from a hair of the test animal of Genus Bos as a template.

6. The method of claim 2, examining the polymorphism in the fifth intron by use of a gene polymorphism detector device.

7. A method for evaluating the amount of the unsaturated fatty acid content in beef fat comprising;
determining a combination of a genotype of sterol regulatory element binding protein (SREBP-1) and a genotype of stearoyl-CoA desaturase (SCD) to evaluate an animal of Genus Bos for better quality of beef with better taste and texture.

8. The method of claim 7, where the animal of Genus Bos having better quality of beef with better taste and texture has a total of three or more alleles when the number of “S type” alleles in the SREBP-1 genotype is combined with the number of “A-type” alleles in the SCD genotype.

9. A bovine-derived SREBP-1 protein, comprising a protein selected from the group consisting of a protein having an amino acid sequence of SEQ NO. 6 and a protein having an amino acid sequence obtained by deletion, replacement, or addition of one or several amino acids in the amino acid sequence of SEQ NO.6 and having a transcriptional regulatory activity by binding a SRE sequence.

10. A bovine-derived SREBP-1 gene, encoding the protein of claim 9.

11. The gene of claim 10, said gene being a DNA selected from the group consisting of a DNA having a base sequence ranging from the 22nd base to the 3324th base, among a base sequence of SEQ NO. 5 and a DNA that has a base sequence hybridized under stringent conditions to a DNA having a base sequence that is complementary to at least 100 base sequence of SEQ NO.5, and that encodes a protein having a transcriptional regulatory activity by binding a SRE sequence.

12. A transgenic cattle, produced by introducing the gene of claim 11.

13. The method of claim 6, where the gene polymorphism detector device is a DNA chip.
